(19) United States
(12) Patent Application Publication
(10) Pub. No.: US 2002/0198202 A1
(43) Pub. Date: Dec. 26, 2002

(54) Combination of a PTPase inhibitor and an antilipemic agent

(75) Inventors: John Thomas Gwynne, Doylestown, PA (US); Philippe John Robert Vitou, Paris (FR)

Correspondence Address: Arnold S. Milowsky, 5 Giralda Farms, Madison, NJ, 07940, US

(73) Assignee: Wyeth, Madison, NJ (US)

(21) Appl. No.: 10/164,231

(22) Filed: Jun. 6, 2002

Related U.S. Application Data
(60) Provisional application No. 60/296,503, filed on Jun. 7, 2001.

(51) Int. Cl. A61K 31/5377; A61K 31/4439; A61K 31/405; A61K 31/381; A61K 31/343

(52) U.S. Cl. 514/232.8; 514/337; 514/339; 514/415; 514/443; 514/469

(57) ABSTRACT

This invention relates to pharmaceutical compositions and methods of treatment utilizing a PTPase (protein-tyrosine phosphatase) inhibitors and an antilipemic agent, such as a bile acid sequestrants, a fibric acid derivative, an HMG-CoA reductase inhibitors, lipase inhibitor or a nicotinic acid derivative, to lower the risk of cardiovascular disease and cardiovascular events in a mammal experiencing or subject to type II diabetes in mammals experiencing or subject to type II diabetes (non-insulin-dependent diabetes mellitus), preferably in human type II diabetics, or in a mammal experiencing or subject to Syndrome X.

This application claims priority from copending provisional application Serial No. 60/296,503, filed Jun. 7, 2001, the entire disclosure of which is hereby incorporated by reference.

This invention relates to pharmaceutical compositions and methods of treatment utilizing a PTPase (protein-tyrosine phosphatase) inhibitors and an antilipemic agent to lower the risk of cardiovascular disease and cardiovascular events in a mammal experiencing or subject to type II diabetes in mammals experiencing or subject to type II diabetes (non-insulin-dependent diabetes mellitus), preferably in human type II diabetics, or in a mammal experiencing or subject to Syndrome X.

### BACKGROUND OF THE INVENTION

The prevalence of insulin resistance in glucose intolerant subjects has long been recognized. Reaven et al (American Journal of Medicine 1976, 60, 80) used a continuous infusion of glucose and insulin (insulin/glucose clamp technique) and oral glucose tolerance tests to demonstrate that insulin resistance existed in a diverse group of nonobese, nonketotic subjects. These subjects ranged from borderline glucose tolerant to overt, fasting hyperglycemia. The diabetic groups in these studies included both insulin dependent (IDDM) and noninsulin dependent (NIDDM) subjects.

Coincident with sustained insulin resistance is the more easily determined hyperinsulinemia, which can be measured by accurate determination of circulating plasma insulin concentration in the plasma of subjects. Hyperinsulinemia can be present as a result of insulin resistance, such as is in obese and/or diabetic (NIDDM) subjects and/or glucose intolerant subjects, or in IDDM subjects, as a consequence of over injection of insulin compared with normal physiological release of the hormone by the endocrine pancreas.

The association of hyperinsulinemia with obesity and with ischemic diseases of the large blood vessels (e.g. atherosclerosis) has been well established by numerous experimental, clinical and epidemiological studies (summarized by Stout, Metabolism 1985, 34, 7, and in more detail by Pyorala et al, Diabetes/Metabolism Reviews 1987, 3, 463). Statistically significant plasma insulin elevations at 1 and 2 hours after oral glucose load correlates with an increased risk of coronary heart disease.

Since most of these studies actually excluded diabetic subjects, data relating the risk of atherosclerotic diseases to the diabetic condition are not as numerous, but point in the same direction as for nondiabetic subjects (Pyorala et al). However, the incidence of atherosclerotic diseases in morbidity and mortality statistics in the diabetic population exceeds that of the nondiabetic population (Pyorala et al; Jarrett Diabetes/Metabolism Reviews 1989,5, 547; Harris et al, Mortality from diabetes, in Diabetes in America 1985).

The independent risk factors obesity and hypertension for atherosclerotic diseases are also associated with insulin resistance. Using a combination of insulin/glucose clamps, tracer glucose infusion and indirect calorimetry, it has been demonstrated that the insulin resistance of essential hypertension is located in peripheral tissues (principally muscle) and correlates directly with the severity of hypertension (DeFronzo and Ferrannini, Diabetes Care 1991, 14, 173). In hypertension of the obese, insulin resistance generates hyperinsulinemia, which is recruited as a mechanism to limit further weight gain via thermogenesis, but insulin also increases renal sodium reabsorption and stimulates the sympathetic nervous system in kidneys, heart, and vasculature, creating hypertension.

It is now appreciated that insulin resistance is usually the result of a defect in the insulin receptor signaling system, at a site post binding of insulin to the receptor. Accumulated scientific evidence demonstrating insulin resistance in the major tissues which respond to insulin (muscle, liver, adipose), strongly suggests that a defect in insulin signal transduction resides at an early step in this cascade, specifically at the insulin receptor kinase activity, which appears to be diminished (reviewed by Haring, Diabetalogia 1991, 34, 848).

Protein-tyrosine phosphatases (PTPases) play an important role in the regulation of phosphorylation of proteins. The interaction of insulin with its receptor leads to phosphorylation of certain tyrosine molecules within the receptor protein, thus activating the receptor kinase. PTPases dephosphorylate the activated insulin receptor, attenuating the tyrosine kinase activity. PTPases can also modulate post-receptor signaling by catalyzing the dephosphorylation of cellular substrates of the insulin receptor kinase. The enzymes that appear most likely to closely associate with the insulin receptor and therefore, most likely to regulate the insulin receptor kinase activity, include PTP1B, LAR, PTPα and SH-PTP2 (B. J. Goldstein, J. Cellular Biochemistry 1992, 48, 33; B. J. Goldstein, Receptor 1993, 3, 1-15,; F. Ahmad and B. J. Goldstein Biochim. Biophys Acta 1995, 1248, 57-69).

McGuire et al. (Diabetes 1991, 40, 939), demonstrated that nondiabetic glucose intolerant subjects possessed significantly elevated levels of PTPase activity in muscle tissue vs. normal subjects, and that insulin infusion failed to suppress PTPase activity as it did in insulin sensitive subjects.

Meyerovitch et al (J. Clinical Invest 1989, 84, 976) observed significantly increased PTPase activity in the livers of two rodent models of IDDM, the genetically diabetic BB rat, and the STZ-induced diabetic rat. Sredy et al (Metabolism, 44, 1074, 1995) observed similar increased PTPase activity in the livers of obese, diabetic ob/ob mice, a genetic rodent model of NIDDM.

The compounds of us in the methods of this invention have been shown to inhibit PTPases derived from rat liver microsomes and human-derived recombinant PTPase-1B (hPTP-1B) in vitro. Their synthesis and use in treatments of insulin resistance associated with obesity, glucose intolerance, diabetes mellitus, hypertension and ischemic diseases of the large and small blood vessels is taught in published PCT Application WO 99/61435 (Wrobel et al.).

### DESCRIPTION OF THE INVENTION

This invention provides pharmaceutical compositions and methods of using PTPase inhibitors in combination with one or more antilipemic agents for improving the cardiovascular risk profile in mammals experiencing or subject to type II diabetes (non-insulin-dependent diabetes mellitus), preferably in human type II diabetics or Syndrome X. These methods may also be characterized as the reduction of risk factors for heart disease, stroke or heart attack in a type II diabetic or a mammal experiencing or subject to Syndrome X.

These methods include the reduction of hyperlipidemia in type II diabetics, including methods in type II diabetics for lowering low density lipoprotein (LDL) blood levels and to increase high density lipoprotein (HDL) blood levels. The methods herein may further be characterized as useful for inhibiting, preventing or reducing atherosclerosis in a type II diabetic or a mammal experiencing or subject to Syndrome X, or the risk factors thereof.

These methods also include the lowering free fatty acid blood levels and triglyceride levels in type II diabetics, or a mammal experiencing or subject to Syndrome X.

Among the antilipemic or agents, also known as antihyperlipidemic agents, which may be utilized with the invention described herein are bile acid sequestrants, fibric acid derivatives, HMG-CoA reductase inhibitors and nicotinic acid compounds. Bile acid sequestrant agents useful with this invention include colestipol and colesevelam, and their pharmaceutically acceptable salt forms. Fibric acid derivatives which may be used with the present invention include clifofibrate, gemfibrozil and fenofibrate. HMG-CoA reductase inhibitors, also known as statins, useful with this invention include cerivastatin, fluvastatin, atorvastatin, lovastatin, pravastatin and simvastatin, or the pharmaceutically acceptable salt forms thereof. Niacin is an example of a nicotinic acid compound which may be used with the methods of this invention. Also useful are lipase inhibiting agents, such as orlistat.

The methods and pharmaceutical compositions of this invention utilize a pharmaceutically effective amount of a PTPase compound of formula I:

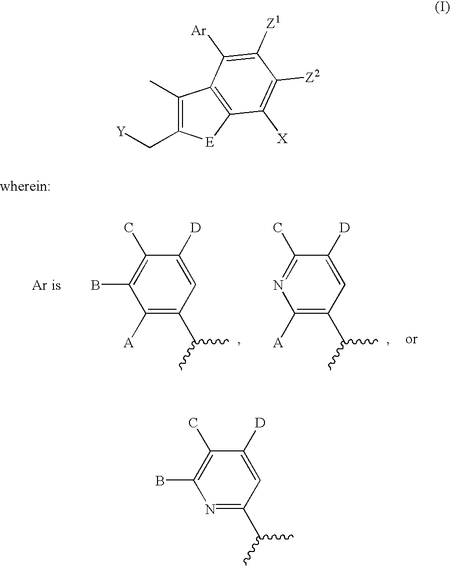

A is hydrogen, halogen, or OH;

B and D are each, independently, hydrogen, halogen, CN, alkyl of 1-6 carbon atoms, aryl, aralkyl of 6-12 carbon atoms, hydroxyalkyl of 1-6 carbon atoms, hydroxyaralkyl of 6-12 carbon atoms, cycloalkyl of 3-8 carbon atoms, nitro, amino, —NR¹R^{1a}, —NR¹COR^{1a}, —NR¹CO₂R^{1a}, cycloalkylamino of 3-8 carbon atoms, morpholino, furan-2-yl, furan-3-yl, thiophen-2-yl, thiophen-3-yl, —COR^{1b} or OR;

R is hydrogen, alkyl of 1-6 carbon atoms, —COR¹, —(CH₂)ₙCO₂R¹, —CH(R^{1a})CO₂R¹, —SO₂R¹, —(CH₂)ₘCH(OH)CO₂R¹, —(CH₂)ₘCOCO₂R¹, —(CH₂)ₘCH═CHCO₂R¹, or —(CH₂)ₘO(CH₂)₀CO₂R¹;

R¹ is hydrogen, alkyl of 1-6 carbon atoms, aralkyl of 6-12 carbon atoms, aryl, or CH₂CO₂R^{1′};

R^{1′} is hydrogen or alkyl of 1-6 carbon atoms

E is S, SO, SO₂, O, or NR^{1c};

X is hydrogen, halogen, alkyl of 1-6 carbon atoms, alkenyl of 2-7 carbon atoms, CN, aryl, aralkyl of 6-12 carbon atoms, hydroxyalkyl of 1-6 carbon atoms, hydroxyaralkyl of 6-12 carbon atoms, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino of 3-8 carbon atoms, morpholino, alkylsulfanyl of 1-6 carbon atoms, arylsulfanyl, pyridylsulfanyl, 2-N,N-dimethylaminoethylsulfanyl, —OCH₂CO₂R^{2b} or —COR^{2c};

Y is hydrogen, halogen, alkyl of 1-6 carbon atoms, aryl, aralkyl of 6-12 carbon atoms, hydroxyalkyl of 1-6 carbon atoms, hydroxyaralkyl of 6-12 carbon atoms, —OR³, SR³, NR³R^{3a}, —COR^{3b}, morpholine or piperidine;

R^{1a}, R^{1c}, R², R^{2a} R³, R^{3a} are each, independently, hydrogen, alkyl of 1-6 carbon atoms, aralkyl of 6-12 carbon atoms, or aryl;

R^{1b} is alkyl of 1-6 carbon atoms or aryl;

R^{2b} is hydrogen, alkyl of 1-6 carbon atoms;

R^{2c} and R^{3b} are each, independently, alkyl of 1-6 carbon atoms, aryl, or aralkyl of 6-12 carbon atoms;

C is hydrogen, halogen or OR⁴;

R⁴ is hydrogen, alkyl of 1-6 carbon atoms, —CH(R₅)W, —C(CH₃)₂CO₂R⁶, 5-thiazolidine-2,4-dione, —CH(R⁷)(CH₂)ₘCO₂R⁶, —COR⁶, —PO₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, or —(CH2)ₚO(CH₂)_{q}CO₂R⁶;

R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), —CH₂(3-pyridyl), —CH₂CO₂H, or —(CH₂)ₙG;

G is NR^{6a}R^{7a}, NR^{6a}COR^{7a},

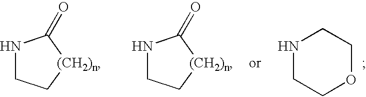

W is CO₂R⁶, CONH₂, CONHOH, CN, CONH(CH₂)₂CN, 5-tetrazole, —PO₃(R⁶)₂, —CH₂OH, —CONR^{6b}CHR^{7b}, —CH₂NR^{6b}CHR^{7b}CO₂R⁶, —CH₂OCHR^{7b}CO₂R⁶—CH₂Br, or —CONR^{6b}CHR^{7b}CO₂R⁶;

R⁶, R^{6a}, R⁷, R^{7a} are each, independently, is hydrogen, alkyl of 1-6 carbon atoms, or aryl;

R^{6b} is hydrogen or —COR^{6c};

R^{6c} is alkyl of 1-6 carbon atoms or aryl;

R^{7b} is hydrogen, alkyl of 1-6 carbon atoms, or hydroxyalkyl of 1-6 carbon atoms;

Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CR¹¹—;

R⁸ is hydrogen, alkyl of 1-6 carbon atoms, or aryl;

R⁹, R¹⁰, and R¹¹ are each, independently, hydrogen, alkyl of 1-6 carbon atoms, aryl, halogen, hydroxy, or alkoxy of 1-6 carbon atoms

m is 1 to 4

n is 1 or 2;

p is 1 to 4;

q is 1 to 4;

or a pharmaceutically acceptable salt or ester form thereof.

The synthesis and PTPase inhibiting and anti-diabetic activities of the compounds described herein are demonstrated in published PCT Application WO 99/61435 (Wrobel et al.), published Dec. 2, 1999, the contents of which are incorporated herein by reference.

Pharmaceutically acceptable salts of these PTPase inhibiting compounds can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety, such as when R⁵ is CH₂(3-pyridyl), or Y is morpholine or contains similar basic moieties. Salts may also be formed from organic and inorganic bases, preferably alkali metal salts, for example, sodium, lithium, or potassium, when a compound of this invention contains a carboxylate or phenolic moiety.

Alkyl includes both straight chain as well as branched moieties. Halogen means bromine, chlorine, fluorine, and iodine. It is preferred that the aryl portion of the aryl or aralkyl substituent is a phenyl or naphthyl; with phenyl being most preferred. The aryl moiety may be optionally mono-, di-, or tri-substituted with a substituent selected from the group consisting of alkyl of 1-6 carbon atoms, alkoxy of 1-6 carbon atoms, trifluoromethyl, halogen, alkoxycarbonyl of 2-7 carbon atoms, alkylamino of 1-6 carbon atoms, and dialkylamino in which each of the alkyl groups is of 1-6 carbon atoms, nitro, cyano, —CO₂H, alkylcarbonyloxy of 2-7 carbon atoms, and alkylcarbonyl of 2-7 carbon atoms.

The PTPase inhibiting compounds used in the methods and compositions of this invention may contain an asymmetric carbon atom and some of the compounds of this invention may contain one or more asymmetric centers and may thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof.

The PTPase inhibiting compounds of this invention may be atropisomers by virtue of possible restricted or slow rotation about the aryl-tricyclic or aryl-bicyle single bond. This restricted rotation creates additional chirality and leads to enantiomeric forms. If there is an additional chiral center in the molecule, diasteriomers exist and can be seen in the NMR and via other analytical techniques. While shown without respect to atropisomer stereochemistry in Formula I, the present invention includes such atoropisomers (enantiomers and diastereomers; as well as the racemic, resolved, pure diastereomers and mixtures of diasteomers) and pharmaceutically acceptable salts thereof.

Preferred PTPase inhibitor compounds of use in this invention include those having the structure:

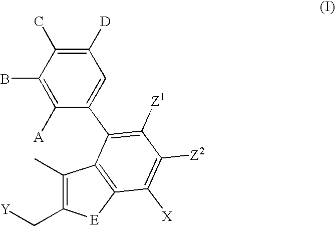

wherein:

A is hydrogen or halogen;

B and D are each, independently, hydrogen, halogen, CN, alkyl of 1-6 carbon atoms, aryl, aralkyl of 6-12 carbon atoms, branched alkyl, cycloalkyl of 3-8 carbon atoms, nitro or OR;

R is hydrogen or alkyl of 1-6 carbon atoms;

E is S, or O;

X is hydrogen, halogen, alkyl of 1-6 carbon atoms, CN, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino, morpholino, alkylsulfanyl of 1-6 carbon atoms, arylsulfanyl, pyridylsulfanyl, 2-N,N-dimethylaminoethylsulfanyl;

R¹, R^{1a}, R², R^{2a}, R³, and R^{3a} are each, independently, hydrogen, alkyl of 1-6 carbon atoms, aralkyl of 6-12 carbon atoms, or aryl;

Y is hydrogen, halogen, OR³, SR³, NR³R^{3a} or morpholine;

C is hydrogen, halogen, or OR⁴;

R⁴ is hydrogen, alkyl of 1-6 carbon atoms, —CH(R⁵)W, —C(CH₃)₂CO₂R⁶, 5-thiazolidine-2,4-dione, —CH(R⁷)(CH₂)ₘCO₂R⁶, —COR⁶, —PO₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, or —(CH₂)ₚO(CH₂)_{q}CO2R⁶;

R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), or —CH₂(3-pyridyl);

W is CO₂R⁶, —CONH₂, —CONHOH, or 5-tetrazole, or —CONR^{6b}CHR^{7b}CO₂R⁶;

R⁶, R^{6a}, R^{6b}, R⁷, R^{7a}, and R^{7b} are each, independently, hydrogen, alkyl of 1-6 carbon atoms, or aryl;

Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CH—;

R⁹ and R¹⁰ are independently, hydrogen, or alkyl of 1-6 carbon atoms;

p is 1 to 4;

q is 1 to 4;

or a pharmaceutically acceptable salt or ester form thereof.

More preferred PTPase inhibiting compounds for use with this invention include those of the structure:

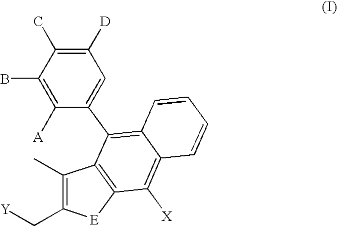

wherein:

A is hydrogen;

B and D are each, independently, halogen, alkyl of 1-6 carbon atoms, aryl, aralkyl of 6-12 carbon atoms, or cycloalkyl of 3-8 carbon atoms;

E is S or O;

X is hydrogen, halogen, alkyl of 1-6 carbon atoms, perfluoroalkyl of 1-6 carbon atoms, CN, alkoxy of 1-6 carbon atoms, aryloxy, arylalkoxy of 6-12 carbon atoms, arylsulfanyl;

Y is hydrogen or —NR¹R², or morpholine;

R¹ and R² are each, independently, hydrogen or alkyl of 1-6 carbon atoms, aralkyl of 6-12 carbon atoms, or aryl;

C is OR⁴;

R⁴ is hydrogen, alkyl of 1-6 carbon atoms, —CH(R⁵)W, or 5-thiazolidine-2,4-dione;

R⁵ is hydrogen, alkyl of 1-6 carbon atoms, aralkyl of 6-12 carbon atoms, aryl, —CH₂(3-1H-indolyl), —CH₂CH₂(1,3-dioxo-1,3-dihydro-isoindol-2-yl), or —CH₂CH₂(1-oxo-1,3-dihydro-isoindol-2-yl);

W is —CO₂R⁶, —CONH₂, —CONHOH, 5-tetrazole, —PO₃(R⁶)₂, or —CONR⁶CHR⁶CO₂R⁶

R⁶ is hydrogen or alkyl of 1-6 carbon atoms;

Z¹ and Z² are taken together as a diene unit having the formula —CH═CH—H═CH—;

or a pharmaceutically acceptable salt thereof.

Even more preferred PTPase inhibitors of this invention include:

(R)-2-[2,6-dibromo-4-(9-bromo-2,3-dimethyl-naphtho[2,3-b]thiophen-4-yl)-phenoxy]-3-phenyl-propionic acid;

(R)-2-[2-bromo-4-(9-bromo-2,3-dimethyl-naphtho[2,3-b]thiophen-4-yl)-6-ethyl-phenoxy]-3-phenyl-propionic acid;

(R)-2-[4-(9-bromo-2,3-dimethyl-naphtho[2,3-b]thiophen-4-yl)-2,6-dimethyl-phenoxy]-3-phenyl-propionic acid;

(R)-2-[4-(9-bromo-2,3-dimethyl-naphtho[2,3-b]thiophen-4-yl)-2-fluoro-phenoxy]-3-phenyl-propionic acid;

[4-(9-bromo-2,3-dimethyl-naphtho[2,3-b]thiophen-4-yl)-2,6-diisopropyl-phenoxy]-acetic acid;

(R)-2-[2-bromo-4-(9-bromo-2,3-dimethyl-naphtho[2,3-b]thiophen-4-yl)-6-sec-butyl-phenoxy]-3-phenyl-propionic acid;

(R)-2-[2-bromo-4-(9-bromo-2,3-dimethyl-naphtho[2,3-b]thiophen-4-yl)-6-isopropyl-phenoxy]-3-phenyl-propionic acid;

(R)-2-[2-bromo-4-(9-bromo-2,3-dimethyl-naphtho[2,3-b]thiophen-4-yl)-2-cyclopentyl-phenoxy]-3-phenyl-propionic acid

(R)-2-[4-(9-bromo-2,3-dimethyl-naphtho[2,3-b]thiophen-4-yl)-6-isopropyl-phenoxy]-3-phenyl-propionic acid;

(R)-2-[4-(9-bromo-2,3-dimethyl-naphtho[2,3-b]thiophen-4-yl)-2-cyclopentyl-phenoxy]-3-phenyl-propionic acid;

(R)-2-[2,6-dibromo-4-(2,3-dimethyl-9-phenylsulfanyl-naphtho[2,3-b]thiophen-4-yl)-phenoxy]-3-phenyl-propionic acid;

(R)-2-[2,6-dibromo-4-(9-bromo-2,3-dimethyl-naphtho[2,3-b]thiophen-4-yl)-phenoxy]-4-phenyl-butyric acid;

(S)-2-[2,6-dibromo-4-(9-bromo-2,3-dimethyl-naphtho[2,3-b]thiophen-4-yl) -phenoxy]-4-phenyl-butyric acid;

2-[2,6-dibromo-4-(9-bromo-3-methyl-2-morpholin-4-ylmethyl-naphtho[2,3-b]thiophen-4-yl)-phenoxy]-3-phenyl-propionic acid;

(R)-2-[2,6-dibromo-4-(2,3-dimethyl-9-phenylsulfanyl-naphtho[2,3-b]thiophen-4-yl)-phenoxy]-propionic acid;

[2-bromo-4-(9-bromo-2,3-dimethyl-naphtho[2,3-b]thiophen-4-yl)-2-nitro-phenoxy]-3-phenyl-propionic acid;

2,6-dibromo-4-(9-bromo-2,3-dimethyl-naphtho[2,3-b]thiophen-4-yl)-phenol;

2-bromo-4-(9-bromo-2,3-dimethyl-naphtho[2,3-b]thiophen-4-yl)-2-nitro-phenol;

(R)-2-[2,6-dibromo-4-(9-bromo-2-diethylaminomethyl-3-methyl-naphtho[2,3-b]-thiophen-4-yl)-phenoxy]-3-phenyl-propionic acid;

(R)-2-[2,6-dibromo-4-(2,3-dimethyl-naphtho[2,3-b]furan-4-yl)-phenoxy]-3-phenyl-propionic acid,

(2R)-2-[4-(9-bromo-2,3-dimethyl-naphtho[2,3-b]thiophen-4-yl)-2,6-diisopropyl-phenoxy]-3-phenyl-propionic acid,

(R)-2-[4-(9-bromo-2-,3-dimethyl-naphtho[2,3-b]thiophen-4-yl)-2,6-diethyl-phenoxy]-3-phenyl-propionic acid,

{(2R)-2-[4-(9-bromo-2,3-dimethyl-naphtho[2,3-b]thiophen-4-yl)-2,6-dimethyl-phenoxy]-3-phenyl-propionylamino}-acetic acid;

{(2R)-2-[4-(9-bromo-2,3-dimethyl-naphtho[2,3-b]thiophen-4-yl)-2,6-diethyl-phenoxy]-3-phenyl-propionylamino}-acetic acid

or pharmaceutically acceptable salts thereof.

Among the most preferred PTPase inhibiting compounds for use in this inventions is (2R)-2-[4-(9-Bromo-2,3-dimethyl-naptho[2,3-b]thiophen-4-yl)-2,6-dimethyl-phenoxy]-3-phenyl-propionic acid, having the structure:

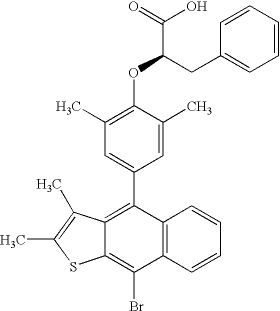

or its pharmaceutically acceptable salt or ester forms.

This invention provides methods for treating, preventing, inhibiting or ameliorating the basis or symptoms of various cardiovascular diseases in a mammal, preferably in a human. This invention provides a number of treatments, therapies or regimens which may be described as methods of using PTPase inhibitors in combination with one more antilipemic agents for improving the cardiovascular risk profile in mammals. This description is intended to describe methods of preventing, inhibiting or reducing or delaying onset of the physiological basis or causative elements of cardiovascular diseases or disorders or likelihood that a recipient would experience an undesirable cardiovascular disease or event associated with type II diabetes.

The methods each comprise administering to a mammal in need thereof a pharmaceutically or therapeutically effective amount of a PTPase inhibitor of this invention, as described herein, and a pharmaceutically or therapeutically effective amount of an antilipemic agent. As used herein a pharmaceutically or therapeutically effective amount is understood to be at least a minimal amount which provides a medical improvement in the symptoms of the specific malady or disorder experienced by the mammal in question. Preferably, the recipient will experience a reduction, inhibition or removal of the biological basis for the malady in question.

A method of this invention comprises a reduction in the risk profile of cardiovascular and cerebrovascular diseases in a mammal. This method may also be described as a method of inhibiting, preventing or reducing the physiological basis or causative elements of cardiovascular diseases. These cardiovascular include atherosclerosis and coronary artery disease.

Another portion of this invention comprises a method of lowering blood cholesterol in a mammal, the method particularly including reduction of lowering of low density lipoprotein (LDL) in a mammal. Also provided is a method of lowering blood triglyceride levels in a mammal. These actions may also be seen as a method for lowering the chances or risk of a mammal experiencing related cardiovascular and cerbrovascular disorders, including coronary artery disease (atherosclerosis), heart attack or stroke.

This invention also comprises methods for-treatment, inhibition or prophylaxis of the cardiovascular conditions mentioned above in a mammal experiencing or subject to Syndrome X and its related and encompassed maladies. These methods include the treatment, inhibition or prevention of hyperlipidemia, hyperglycemia, hypertension, cardiovascular and cerebrovascular disease, including atherosclerosis, and renal disease associated with Syndrome X. Also included in these methods are the treatments described above in a mammal currently experiencing or subject to symptoms or conditions of Syndrome X, including diabetic neuropathy, microalbuminaria, albuminaria, glomerular sclerosis, glomerulonephritis, nephrotic syndrome, end stage renal disease and hypertensive nephrosclerosis. As described herein, each of these methods for improving the cardiovascular or cerebrovascular risk profiles and for lowering the risk of cardiovascular disease and cardiovascular events in a mammal experiencing or subject to Syndrome X comprises administering to a mammal in need of such treatment a pharmaceutically effective amount of a PTPase inhibitor of this invention, or a pharmaceutically acceptable salt form thereof, and a pharmaceutically effective amount of an antilipemic agent.

Another aspect of this invention comprises a pharmaceutical composition comprising a pharmaceutically effective amount of a PTPase inhibitor compound of this invention, a pharmaceutically effective amount of an antilipemic agent and one or more pharmaceutically acceptable carriers or excipients.

Effective administration of the PTPase inhibitor compounds herein may be given at a daily dosage of from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations herein may utilize standard delay or time release formulations to alter the absorption of the active compound(s). Suppository formulations may be made from traditional materials, including cocoa butter, with or without the addition of waxes to alter the suppository's melting point, and glycerin. Water soluble suppository bases, such as polyethylene glycols of various molecular weights, may also be used.

Antilipemic agents which are useful in the methods and combinations of this invention include bile acid sequestrants, fibric acid derivatives, HMG-CoA reductase inhibitors, nicotinic acid compounds or lipase inhibiting agents. Representative agents for each of these groups are known and may be administered in doses and regimens known in the art. Examples of the doses and administration regimens for these compounds can be seen in the Physicians'Desk Reference, 55 Edition, 2001, published by Medical Economics Company, Inc. at Montvale, N.J., the relevant sections of which are incorporated herein by reference.

Bile acid sequestrant agents useful with this invention include colestipol and colesevelam, and their pharmaceutically acceptable salt forms. Colestipol is available in 1 mg COLESTID® micronized colestipol hydrochloride tablets from Pharmacia & Upjohn, with a recommended initial dose of about 2 g per day, which may be increased as need to a dose of from 2 to 16 g per day taken in divided doses. Colesevelam hydrochloride is available in 625 mg WELCHOL™ tablets from Sankyo Pharma, Inc., with a recommended starting dose of 3 tablets taken twice per day with meals or 6 tablets taken once per day with a meal. If needed, the administration may be increased to 7 tablets per day. Administration of tablets with liquid is recommended.

Fibric acid derivatives which may be used with the present invention include clifofibrate, gemfibrozil and fenofibrate. Clifofibrate is commercially available in the form of 500 mg ATROMID-S® capsules from Wyeth-Ayerst Pharmaceuticals, with a recommended daily dosage of about 2 g administered in divided doses. Gemfibrozoil is available in 600 mg LOPID® tablets from Parke-Davis, with a recommended dose for adults of about 1200 mg per day administered in two divided doses 30 minutes prior to the morning and evening meals. Fenofibrate is available in 67 mg, 134 mg and 200 mg TRICOR® tablets from Abbott Laboratories Inc., with a recommended initial dose of from 67 mg to 200 mg per day, up to a maximum daily dose of 200 mg per day.

HMG-CoA reductase inhibitors useful with this invention include cerivastatin, fluvastatin, atorvastatin, lovastatin, pravastatin and simvastatin, or the pharmaceutically acceptable salt forms thereof. BAYCOL® cerivastatin sodium tablets in 0.2 mg, 0.3 mg, 0.4 mg and 0.8 mg tablet doses are available from Bayer Corporation, with a recommended starting dose of 0.4 mg taken once daily in the evening, with a maintenance dosage range of from 0.2 mg to 0.8 mg per day. LESCOL® fluvastatin sodium capsules containing fluvastatin sodium equivalent to 20 mg or 40 mg fluvastatin are available from Novartis Pharmaceuticals Corporation with a recommended starting dose of 20 mg to 40 mg taken once daily at bedtime, and a recommended daily maintenance dose of from 20 mg to 80 mg, with a daily dose of 80 mg being taken in divided doses. LIPITOR® Atorvastatin calcium tablets are available in 10 mg, 20 mg, 40 mg or 80 mg doses from Park Davis or Pfizer Inc., with a recommended starting dose of 10 mg taken once daily, with a final dosage range of from 10 mg to 80 mg once daily. MEVACOR® lovastatin tablets are available in 10 mg, 20 mg and 40 mg tablets from Merck & Co., Inc., with a recommended starting dose of 20 mg taken once daily with the evening meal and a recommended dosing range of from 10 mg to 80 mg per day in a single or two divided doses. PRAVACHOL® pravastatin sodium tablets are available from Bristol-Meyers Squibb Company as 10 mg, 20 mg or 40 mg tablets, with a recommended starting dose of 10 mg, 20 mg or 40 mg taken once daily. ZOCOR® simvastatin tablets are available in 5 mg, 10 mg, 20 mg, 40 mg or 80 mg doses from Merck & Co., with a recommended starting dose of 20 mg per day and a maintenance dosage range of from 5 mg to 80 mg per day.

Niacin is an example of a nicotinic acid agent which may be used with the methods of this invention. It is commercially available in 500 mg, 750 mg and 1,000 mg extended release tablets under the NIASPAN® tradename from Kos Pharmaceuticals, Inc., 1001 Brickell Bay Drive, 25^{th} Floor, Miami, Fla. 33131.

Orlistat is a lipase inhibiting agent available in 120 mg capsules under the XENICAL® tradename from Roche Pharmaceuticals. Recommended dosage is one 120 mg tablet three times per day after each main meal containing fat.

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved. It is also preferred that the recipient also utilize art recognized lifestyle patterns for reducing the incidence of the maladies described herein. These include maintenance of an appropriate diet and exercise regimen, as recommended by a medical practitioner familiar with the physical condition of the recipient.

The following are representative PTPase inhibiting compound examples useful in the compositions and methods of this invention. Their synthesis is described in published PCT Application WO 99/61435, published Dec. 2,1999, the contents of which are incorporated herein by reference.

### EXAMPLE 1

2,3-Dimethyl-thiophene;

### EXAMPLE 2

4,5-Dimethylthiophene-2-yl-(phenyl)-methanol;

### EXAMPLE 3

2-Benzyl-4,5 dimethylthiophene;

### EXAMPLE 4

(2-Benzyl-4,5-dimethyl-thiophen-3-yl)-(4-methoxy-phenyl)-methanone;

### EXAMPLE 5

4-(2,3-Dimethyl-naphtho[2,3-b]thiophen-4-yl-phenol;

### EXAMPLE 6

Acetic Acid 4-(2,3-dimethyl-naphtho[2,3-b]thiophen-4-yl)-phenyl ester;

### EXAMPLE 7

Acetic Acid 4-(9-bromo-2,3-dimethyl-naphtho[2,3-b]thiophen-4-yl)-phenyl ester;

### EXAMPLE 8

4-(9-Bromo-2,3-dimethyl-naphtho[2,3-b]thiophen-4-yl)-phenol;

### EXAMPLE 9

2,6-Dibromo-4-(9-bromo-2,3-dimethyl-naphtho[2,3-b]thiophen-4-yl)-phenol;

### EXAMPLE 10

Methanesulfonic acid 4-(2,3-dimethyl-naphtho[2,3-b]thiophen-4-yl)-phenyl ester;

### EXAMPLE 11

Methanesulfonic acid 4-(9-iodo-2,3-dimethyl-naphtho[2,3-b]thiophen-4-yl)-phenyl ester;

### EXAMPLE 12

4-(2,3-Dimethyl-9-phenylsulfanyl-naphtho[2,3-b]thiophen-4-yl)-phenol;

### EXAMPLE 13

2,6-Dibromo-4-(2,3-dimethyl-9-phenylsulfanyl-naphtho[2,3-b]thiophen-4-yl)-phenol;

### EXAMPLE 14

Acetic acid 4-(9-bromo-2-chloromethyl-3-methyl-naphtho[2,3-b]thiophen-4-yl)-phenyl ester;

### EXAMPLE 15

4-(9-Bromo-3-methyl-2-morpholin-4-yl)methyl-naphtho[2,3-b]thiophen-4-yl)-phenol;

### EXAMPLE 16

4-(9-Bromo-2-diethylaminomethyl-3-methyl-naphtho[2,3-b]thiophen-4-yl)-acetate;

### EXAMPLE 17

4-(9-Bromo-2-diethylaminomethyl-3-methyl-naphtho[2,3-b]thiophen-4-yl)-phenol;

### EXAMPLE 18

2,6-Dibromo-4-(9-bromo-2-diethylaminomethyl-3-methyl-naphtho[2,3-b]thiophen-4-yl)-phenol;

### EXAMPLE 19

2,6-Dibromo-4-(9-bromo-3-methyl-2-morpholin-4-ylmethyl-naphtho [2,3-b]thiophen-4-yl)-phenol;

### EXAMPLE 20

4-(9-Bromo-2,3-dimethyl-naphtho[2,3-b]thiophen-4-yl)-2-nitro-phenol;

### EXAMPLE 21

2-Bromo-4-(9-bromo-2,3-dimethyl-naphtho[2,3-b]thiophen-4-yl)-6-nitro-phenol;

### EXAMPLE 22

2-Amino-4-(9-bromo-2,3-dimethyl-naphtho[2,3-b]thiophen-4-yl)-phenol;

### EXAMPLE 23

2-Amino-6-bromo-4-(9-bromo-2,3-dimethyl-naphtho[2,3-b]thiophen-4-yl)-phenol;

### EXAMPLE 24

[2-Bromo-4-(9-bromo-2,3-dimethyl-naphtho[2,3-b]thiophen-4-yl)-2-nitro-phenoxy]-acetic acid;

### EXAMPLE 25

(S)-2-Hydroxy-3-phenylpropionic acid, methyl ester;

### EXAMPLE 26

(S)-2-[4-Nitrobenzoyl]-4-phenylbutyric acid, ethyl ester;

### EXAMPLE 27

(S)-2-Hydroxy-4-phenylbutyric Acid, ethyl ester;

### EXAMPLE 28

(R)-2-[2,6-Dibromo-4-(9-bromo-2,3-dimethyl-naphtho[2,3-b]thiophen-4-yl)-phenoxy]3-phenyl-propionic acid methyl ester;

### EXAMPLE 29

(R)-2-[2,6-Dibromo-4-(9-bromo-2,3-dimethyl-naphtho[2,3-b]thiophen-4-yl)-phenoxy]3-phenyl-propionic acid;

### EXAMPLE 30

(R)-2-[2,6-Dibromo-4-(9-bromo-2,3-dimethylnaptho[2,3-b]thien-4-yl)-phenoxy]-propanoic acid;

### EXAMPLE 31

(S)-2-[2,6-Dibromo-4-(9-bromo-2,3-dimethyl-naphtho[2,3-b]thiophen-4-yl)-phenoxy]-4-phenyl-butyric acid;

### EXAMPLE 32

(R)-2-[2,6-Dibromo-4-(9-bromo-2,3-dimethyl-naphtho[2,3-b]thiophen-4-yl)-phenoxy]-4-phenyl-butyric acid;

### EXAMPLE 33

(R)-2-[2,6-dibromo-4-(2,3-dimethyl-9-phenylsulfanyl-naphtho[2,3-b]-thiophen-4-yl)-phenoxy]-3-phenyl-propionic acid;

### EXAMPLE 34

(R)-2-[2,6-Dibromo-4-(2,3-dimethyl-9-phenylsulfanyl-naphtho[2,3-b]-thiophen-4-yl)-phenoxy]-propionic acid;

### EXAMPLE 35

2-[2,6-Dibromo-4-(9-bromo-3-methyl-2-morpholin-4-ylmethyl-naphtho-[2,3-b]thiophen-4-yl)-phenoxy]-3-phenyl-propionic acid;

### EXAMPLE 36

2-[2,6-Dibromo-4-(9-bromo-3-methyl-2-morpholin-4-ylmethyl-naphtho-[2,3-b]thiophen-4-yl)-phenoxy]-propionic acid;

### EXAMPLE 37

(R)-2-[2,6-Dibromo-4-(9-bromo-2-diethylaminomethyl-3-methyl-naphtho[2,3-b]thiophen-4-yl)-phenoxy]-3-phenyl-propionic acid;

### EXAMPLE 38

[2-Bromo-4-(9-bromo-2,3-dimethyl-naphtho[2,3-b]thiophen-4-yl)-2-nitro-phenoxy]-3-phenyl-propionic acid;

### EXAMPLE 39

2-Bromo-4-(9-bromo-2,3-dimethyl-naphtho[2,3-b]thiophen-4-yl)-6-isopropyl-phenol;

### EXAMPLE 40

(R)-2-[2-Bromo-4-(9-bromo-2,3-dimethyl-naphtho[2,3-b]thiophen-4-yl)-6-isopropyl-phenoxy]-3-phenyl-propionic acid;

### EXAMPLE 41

(R)-2-[4-(2,3-dimethyl-naphtho[2,3-b]thiophen-4-yl)-2-isopropyl-phenoxy]-3-phenyl-propionic acid;

### EXAMPLE 42

(R)-2-[2-Bromo-4-(9-bromo-2,3-dimethyl-naphtho[2,3-b]thiophen-4-yl)-6-sec-butyl-phenoxy]-3-phenyl-propionic acid;

### EXAMPLE 43

(R)-2-[2-Bromo-4-(9-bromo-2,3-dimethyl-naphtho[2,3-b]thiophen-4-yl)-6-ethyl-phenoxy]-3-phenyl-propionic acid;

### EXAMPLE 44

(R)-2-[4-(9-Bromo-2,3-dimethyl-naphtho[2,3-b]thiophen-4-yl)-6-isopropyl-phenoxy]-3-phenyl-propionic acid;

### EXAMPLE 45

(R)-2-[2-Cyclopentyl-4-(2,3-dimethyl-naphtho[2,3-b]thiophen-4-yl)-phenoxy]-3-phenyl-propionic acid;

### EXAMPLE 46

(R)-2-[4-(2,3-Dimethyl-naphtho[2,3-b]thiophen-4-yl)-2,6-dimethyl-phenoxy]-3-phenyl-propionic acid;

### EXAMPLE 47

R)-2-[4-(9-Bromo-2,3-dimethyl-naphtho[2,3-b]thiophen-4-yl)-2-cyclopentyl-phenoxy]-3-phenyl-propionic acid;

### EXAMPLE 48

(R)-2-[2-Bromo-4-(9-bromo-2,3-dimethyl-naphtho[2,3-b]thiophen-4-yl)-2-cyclopentyl-phenoxy]-3-phenyl-propionic acid;

### EXAMPLE 49

(R)-2-[4-(9-Bromo-2,3-dimethyl-naphtho[2,3-b]thiophen-4-yl)-2,6-dimethyl-phenoxy]-3-phenyl-propionic acid;

### EXAMPLE 50

(R)-2-[4-(2,3-Dimethyl-naphtho[2,3-b]thiophen-4-yl)-2,6-diisopropyl-phenoxy]-3-phenyl-propionic acid;

### EXAMPLE 51

(R)-2-[4-(2,3-Dimethyl-naphtho[2,3-b]thiophen-4-yl)-2-fluoro-phenoxy]-3-phenyl-propionic acid;

### EXAMPLE 52

(R)-2-[4-(9-Bromo-2,3-dimethyl-naphtho[2,3-b]thiophen-4-yl)-2-fluoro-phenoxy]-3-phenyl-propionic acid;

### EXAMPLE 53

[4-(9-Bromo-2,3-dimethyl-naphtho[2,3-b]thiophen-4-yl)-2,6-diisopropyl-phenoxy]-acetic acid;

### EXAMPLE 54

(2R)-2-[2,6-Dibromo-4-(2,3-dimethyl-naphtho[2,3-b]furan-4-yl)-phenoxy]-3-phenyl-propionic acid;

### EXAMPLE 55

(2R)-2-[4-(9-Bromo-2,3-dimethyl-naphtho[2,3-b]thiophen-4-yl)-2,6-diisopropyl-phenoxy]-3-phenyl-propionic acid;

### EXAMPLE 56

[3-Bromo-5-(9-bromo-2,3-dimethyl-naphtho[2,3-b]thiophen-4-yl)-2-hydroxy-phenyl]-carbamic acid tert-butyl ester;

### EXAMPLE 57

9-Bromo-4-(3-bromo-methoxy-5-nitro-phenyl)-2,3-dimethyl-naphtho-[2,3-b]thiophene;

### EXAMPLE 58

3-Bromo-5-(9-bromo-2,3-dimethyl-naphtho[2,3-b]thiophen-4-yl)-2-methoxy-phenylamine;

### EXAMPLE 59

[3-Bromo-5-(9-bromo-2,3-dimethyl-naphtho[2,3-b]thiophen4-yl)-2-methoxy-phenylamino]-acetic acid methyl ester;

### EXAMPLE 60

[3-Bromo-5-(9-bromo-2,3-dimethyl-naphtho[2,3-b]thiophen4-yl)-2-methoxy-phenylamino]-acetic acid;

### EXAMPLE 61

(R)-2-[4-(9-Bromo-2-,3-dimethyl-naphtho[2,3-b]thiophen-4-yl)-2,6-diethyl-phenoxy]-3-phenyl-propionic acid;

### EXAMPLE 62

{(2R)-2-[4-(9-Bromo-2,3-dimethyl-naphtho[2,3-b]thiophen-4-yl)-2,6-dimethyl-phenoxy]-3-phenyl-propionylamino}-acetic acid;

### EXAMPLE 63

{(2R)-2-[4-(9-Bromo-2,3-dimethyl-naphtho[2,3-b]thiophen-4-yl)-2,6-diethyl-phenoxy]-3-phenyl-propionylamino}-acetic acid;

### EXAMPLE 64

(2R)-2-[4-(9-Bromo-2,3-dimethyl-naphtho[2,3-b]thiophen-4-yl) -phenoxy]-3-phenyl-propionic acid;

### EXAMPLE 65

(2S)-2-[4-(9-Bromo-2-,3-dimethyl-naphtho[2,3-b]thiophen-4-yl)-2,6dimethyl-phenoxy]-3-phenyl-propionic acid;

### EXAMPLE 66

(2R)-2-[4-(9-Bromo-2,3-dimethyl-1-oxo-1H-naphtho[2,3-b]thiophen-4-yl)-2,6-dimethyl-phenoxy]-3-phenyl-propionic acid;

### EXAMPLE 67

(R)-2-[4-(2-,3-Dimethyl-naphtho[2,3-b]thiophen-4-yl)-2,6-diethyl-phenoxy]-3-phenyl-propionic acid;

### EXAMPLE 68

{(2R)-2-[4-(2,3-Dimethyl-naphtho[2,3-b]thiophen-4-yl)-2,6-diethyl-phenoxy]-3-phenyl-propionylamino}-acetic acid;

### EXAMPLE 69

4-(2,3-Dimethyl-naphtho[2,3-b]furan-4-yl)-2,6-diethyl-phenol;

### EXAMPLE 70

(R)-2-[4-(9-Bromo-2-,3-dimethyl-naphtho[2,3-b]furan-4-yl)-2,6-diethyl-phenoxy]-3-phenyl-propionic acid;

### EXAMPLE 71

(R)-2-[2-Cyclopentyl-4-(2-,3-dimethyl-naphtho[2,3-b]thiophen-4-yl)-phenoxy]-propionic acid;

### EXAMPLE 72

(R)-2-[4-(9-Bromo-2-,3-dimethyl-naphtho[2,3-b]thiophen-4-yl)-2-cyclopentyl-phenoxy]-propionic acid;

### EXAMPLE 73

4-[4-(9-Bromo-2-,3-dimethyl-naphtho[2,3-b]thiophen-4-yl)-2-cyclopentyl-phenoxy]-butyric acid;

### EXAMPLE 74

2-Cyclopentyl-4-(2-,3-dimethyl-naphtho[2,3-b]furan-4-yl)-phenol;

### EXAMPLE 75

Acetic acid 2-cyclopentyl-4-(2-,3-dimethyl-naphtho[2,3-b]furan-4-yl)-phenyl ester;

### EXAMPLE 76

(R)-2-[4-(2-,3-Dimethyl-naphtho[2,3-b]thiophen-4-yl)-2-ethyl-phenoxy]-3-phenyl-propionic acid;

### EXAMPLE 77

(R)-2-[4-(9-Bromo-2-,3-dimethyl-naphtho[2,3-b]thiophen-4-yl)-2-ethyl-phenoxy]-3-phenyl-propionic acid;

### EXAMPLE 78

2-Bromo-4-(2-,3-dimethyl-naphtho[2,3-b]furan-4-yl)-6-ethyl-phenol;

### EXAMPLE 79

(R)-2-[2-Bromo-4-(2-,3-dimethyl-naphtho[2,3-b]furan-4-yl)-6-ethyl-phenoxy]-3-phenyl-propionic acid;

### EXAMPLE 80

4-[2-Bromo-4-(2,3-dimethyl-naphtho[2,3-b]furan-4-yl)-6-ethyl-phenoxy]-butyric acid;

### EXAMPLE 81

4-[2-Bromo-4-(2,3-dimethyl-naphtho[2,3-b]furan-4-yl)-6-ethyl -phenoxy]-butyramide 0.4 hydrate;

### EXAMPLE 82

4-(2,3-Dimethyl-naphtho[2,3-b]furan-4-yl)-2-ethyl-phenol;

### EXAMPLE 83

(R)-2-[4-(9-Bromo-2-,3-dimethyl-naphtho[2,3-b]thiophen-4-yl)-2-propyl -phenoxy]-3-phenyl-propionic acid;

### EXAMPLE 84

[9-Bromo-4-(4-methoxy-3,5-dimethylphenyl)-3-methylnaphtho[2,3-b]-thien-2-yl]methyl acetate;

### EXAMPLE 85

4-(9-Bromo-2-,3-dimethyl-naphtho[2,3-b]thien-4-yl)-2-methyl-phenyl acetate;

### EXAMPLE 86

Acetic acid 4-(9-bromo-2-diethylaminomethyl-3-methyl-naphtho[2,3-b]-thiophen-4-yl)-2,6-dimethyl-phenyl ester;

### EXAMPLE 87

2-[4-(9-Bromo-2-diethylaminomethyl-3-methyl-naphtho[2,3-b]thiophen -4-yl)-2,6-dimethyl-phenoxy]-3-phenyl-propionic acid; and

### EXAMPLE 88

(2R)-2-[4-(9-Bromo-2-diethylaminomethyl-3-methyl-naphtho[2,3-b]-thiophen-4-yl)-2,6-diisopropyl-phenoxy]-3-phenyl-propionic acid;

or the pharmaceutically acceptable salt or ester forms thereof.

What is claimed:

1. A method for improving the cardiovascular risk profile in a mammal experiencing or subject to Syndrome X or type II diabetes, the method comprising administering to a mammal in need thereof a pharmaceutically effective amount of an antilipemic agent and a pharmaceutically effective amount of a protein-tyrosine phosphatase inhibitor compound of the formula: 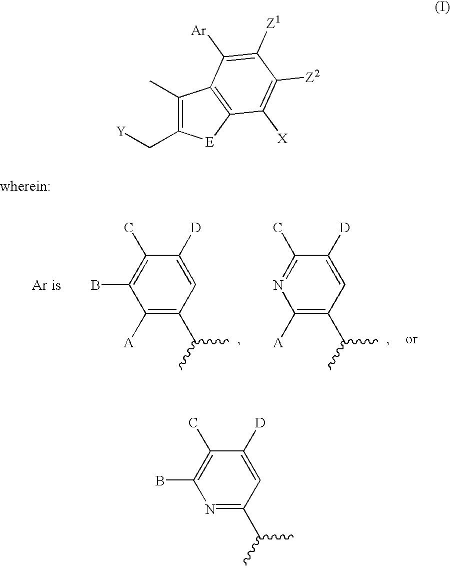
A is hydrogen, halogen, or OH;
B and D are each, independently, hydrogen, halogen, CN, alkyl of 1-6 carbon atoms, aryl, aralkyl of 6-12 carbon atoms, hydroxyalkyl of 1-6 carbon atoms, hydroxyaralkyl of 6-12 carbon atoms, cycloalkyl of 3-8 carbon atoms, nitro, amino, —NR¹R^{1a}, —NR¹COR^{1a}, —NR¹CO₂R^{1a}, cycloalkylamino of 3-8 carbon atoms, morpholino, furan-2-yl, furan-3-yl, thiophen-2-yl, thiophen-3-yl, —COR^{1b} or OR;
R is hydrogen, alkyl of 1-6 carbon atoms, —COR¹, —(CH₂)ₙCO₂R¹, —CH(R^{1a})CO₂R¹, —SO₂R¹, —(CH₂)ₘCH(OH)CO₂R¹, —(CH₂)ₘCOCO₂R¹, —(CH₂)ₘCH═CHCO₂R¹, or —(CH₂)ₘO(CH₂)ₒCO₂R¹;
R¹ is hydrogen, alkyl of 1-6 carbon atoms, aralkyl of 6-12 carbon atoms, aryl, or CH₂CO₂R^{1′};
R^{1′} is hydrogen or alkyl of 1-6 carbon atoms
E is S, SO, SO₂, O, or NR^{1c};
X is hydrogen, halogen, alkyl of 1-6 carbon atoms, alkenyl of 2-7 carbon atoms, CN, aryl, aralkyl of 6-12 carbon atoms, hydroxyalkyl of 1-6 carbon atoms, hydroxyaralkyl of 6-12 carbon atoms, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino of 3-8 carbon atoms, morpholino, alkylsulfanyl of 1-6 carbon atoms, arylsulfanyl, pyridylsulfanyl, 2-N,N-dimethylaminoethyl-sulfanyl, —OCH₂CO₂R^{2b} or —COR^{2c};
Y is hydrogen, halogen, alkyl of 1-6 carbon atoms, aryl, aralkyl of 6-12 carbon atoms, hydroxyalkyl of 1-6 carbon atoms, hydroxyaralkyl of 6-12 carbon atoms, —OR³, SR³, NR³R^{3a}, —COR^{3b}, morpholine or piperidine;
R^{1a}, R^{1c}, R², R^{2a} R³, R^{3a} are each, independently, hydrogen, alkyl of 1-6 carbon atoms, aralkyl of 6-12 carbon atoms, or aryl;
R^{1b} is alkyl of 1-6 carbon atoms or aryl;
R^{2b} is hydrogen, alkyl of 1-6 carbon atoms;
R^{2c} and R^{3b} are each, independently, alkyl of 1-6 carbon atoms, aryl, or aralkyl of 6-12 carbon atoms;
C is hydrogen, halogen or OR⁴;
R⁴ is hydrogen, alkyl of 1-6 carbon atoms, —CH(R₅)W, —C(CH₃)₂CO₂R⁶, 5-thiazolide-2,4-dione, —CH(R⁷)(CH₂)ₘCO₂R⁶, —COR⁶, —PO₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, or —(CH2)ₚO(CH₂)_{q}CO₂R⁶;
R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), —CH₂(3-pyridyl), —CH₂CO₂H, or —(CH₂)ₙG;
G is NR^{6a}R^{7a}, NR^{6a}COR^{7a}, 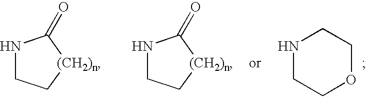
W is CO₂R⁶, CONH₂, CONHOH, CN, CONH(CH₂)₂CN, 5-tetrazole, —PO₃(R⁶)₂, —CH₂OH, —CONR^{6b}CHR^{7b}, —CH₂NR^{6b}CHR^{7b}CO₂R⁶, —CH₂OCHR^{7b}CO₂R⁶ —CH₂Br, or —CONR^{6b}CHR^{7b}CO₂R⁶;
R⁶, R^{6a}, R⁷, R^{7a} are each, independently, is hydrogen, alkyl of 1-6 carbon atoms, or aryl;
R^{6b} is hydrogen or —COR^{6c};
R^{6c} is alkyl of 1-6 carbon atoms or aryl;
R^{7b} is hydrogen, alkyl of 1-6 carbon atoms, or hydroxyalkyl of 1-6 carbon atoms;
Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CR¹¹—;
R⁸ is hydrogen, alkyl of 1-6 carbon atoms, or aryl;
R⁹, R¹⁰, and R¹¹ are each, independently, hydrogen, alkyl of 1-6 carbon atoms, aryl, halogen, hydroxy, or alkoxy of 1-6 carbon atoms
m is 1 to 4
n is 1 or 2;
p is 1 to 4;
q is 1 to 4;
or a pharmaceutically acceptable salt thereof.

2. A method according to claim 1, wherein
Ar is 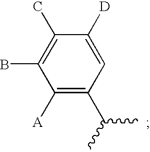
A is hydrogen or halogen
B and D are each, independently, hydrogen, halogen, CN, alkyl of 1-6 carbon atoms, aryl, aralkyl of 6-12 carbon atoms, branched alkyl, cycloalkyl of 3-8 carbon atoms, nitro or OR;
R is hydrogen or alkyl of 1-6 carbon atoms;
E is S, or O;
X is hydrogen, halogen, alkyl of 1-6 carbon atoms, CN, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino, morpholino, alkylsulfanyl of 1-6 carbon atoms, arylsulfanyl, pyridylsulfanyl, or 2-N,N-dimethylaminoethylsulfanyl;
R¹, R^{1a}, R², R^{2a}, R³, and R^{3a} are each, independently, hydrogen, alkyl of 1-6 carbon atoms, aralkyl of 6-12 carbon atoms, or aryl;
Y is hydrogen, halogen, OR³, SR³, NR³R^{3a}, or morpholine;
C is hydrogen, halogen, or OR⁴;
R⁴ is hydrogen, alkyl of 1-6 carbon atoms, —CH(R⁵)W, —C(CH₃)₂CO₂R⁶, 5-thiazolidine-2,4-dione, —CH(R⁷)(CH₂)ₘCO₂R⁶, —COR⁶, —PO₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, —(CH²)ₚO(CH²)_{q}CO₂R⁶;
R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), or —CH₂(3-pyridyl);
W is CO₂R⁶, —CONH₂, —CONHOH, 5-tetrazole, or —CONR^{6b}CHR^{7b}CO₂R⁶;
R⁶, R^{6a}, R^{6b}, R⁷, R^{7a}, and R^{7b} are each, independently, hydrogen, alkyl of 1-6 carbon atoms, or aryl;
Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CH—;
R⁹ and R¹⁰ are each, independently, hydrogen, or alkyl of 1-6 carbon atoms;
p is 1 to 4;
q is 1 to 4;
or a pharmaceutically acceptable salt or ester form thereof.

3. A method according to claim 1, wherein
A is hydrogen;
B and D are each, independently, halogen, alkyl of 1-6 carbon atoms, aryl, aralkyl of 6-12 carbon atoms, or cycloalkyl of 3-8 carbon atoms;
E is S or O;
X is hydrogen, halogen, alkyl of 1-6 carbon atoms, perfluoroalkyl of 1-6 carbon atoms, CN, alkoxy of 1-6 carbon atoms, aryloxy, arylalkoxy of 6-12 carbon atoms, arylsulfanyl;
Y is hydrogen, —NR¹R², or morpholine;
R¹ and R² are each, independently, hydrogen or alkyl of 1-6 carbon atoms, aralkyl of 6-12 carbon atoms, or aryl;
C is OR⁴;
R⁴ is hydrogen, alkyl of 1-6 carbon atoms, —CH(R⁵)W, or 5-thiazolidine-2,4-dione;
R⁵ is hydrogen, alkyl of 1-6 carbon atoms, aralkyl of 6-12 carbon atoms, aryl, —CH₂(3-1H-indolyl), —CH₂CH₂(1,3-dioxo-1,3-dihydro-isoindol-2-yl), or —CH₂CH₂(1-oxo-1,3-dihydro-isoindol-2-yl);
W is —CO₂R⁶, —CONH₂, —CONHOH, 5-tetrazole, —PO₃(R⁶)₂, or —CONR⁶CHR⁶CO₂R⁶;
R⁶ is hydrogen or alkyl of 1-6 carbon atoms;
Z¹ and Z² are taken together as a diene unit having the formula —CH═CH—H═CH—;
or a pharmaceutically acceptable salt or ester form thereof.

4. A method according to claim 1 wherein the protein-tyrosine phosphatase inhibitor compound is selected from the group of
(R)-2-[2,6-dibromo-4-(9-bromo-2,3-dimethyl-naphtho[2,3-b]thiophen-4-yl)-phenoxy]-3-phenyl-propionic acid;
(R)-2-[2-bromo-4-(9-bromo-2,3-dimethyl-naphtho[2,3-b]thiophen-4-yl)-6-ethyl-phenoxy]-3-phenyl-propionic acid;
(R)-2-[4-(9-bromo-2,3-dimethyl-naphtho[2,3-b]thiophen-4-yl)-2,6-dimethyl-phenoxy]-3-phenyl-propionic acid;
(R)-2-[4-(9-bromo-2,3-dimethyl-naphtho[2,3-b]thiophen-4-yl)-2-fluoro-phenoxy]-3-phenyl-propionic acid; or
[4-(9-bromo-2,3-dimethyl-naphtho[2,3-b]thiophen-4-yl)-2,6-diisopropyl-phenoxy]-acetic acid; or a pharmaceutically acceptable salt or ester form thereof.

5. A method according to claim 1 wherein the protein-tyrosine phosphatase inhibitor compound is selected from the group of:
(R)-2-[2-bromo-4-(9-bromo-2,3-dimethyl-naphtho[2,3-b]thiophen-4-yl)-6-sec-butyl-phenoxy]-3-phenyl-propionic acid;
(R)-2-[2-bromo-4-(9-bromo-2,3-dimethyl-naphtho[2,3-b]thiophen-4-yl)-6-isopropyl-phenoxy]-3-phenyl-propionic acid;
(R)-2-[2-bromo-4-(9-bromo-2,3-dimethyl-naphtho[2,3-b]thiophen-4-yl)-2-cyclopentyl-phenoxy]-3-phenyl-propionic acid;
(R)-2-[4-(9-bromo-2,3-dimethyl-naphtho[2,3-b]thiophen-4-yl)-6-isopropyl-phenoxy]-3-phenyl-propionic acid; or
(R)-2-[4-(9-bromo-2,3-dimethyl-naphtho[2,3-b]thiophen-4-yl)-2-cyclopentyl-phenoxy]-3-phenyl-propionic acid; or a pharmaceutically acceptable salt or ester form thereof.

6. A method according to claim 1 wherein the protein-tyrosine phosphatase inhibitor compound is selected from the group of:
(R)-2-[2,6-dibromo-4-(2,3-dimethyl-9-phenylsulfanyl-naphtho[2,3-b]thiophen-4-yl)-phenoxy]-3-phenyl-propionic acid;
(R)-2-[2,6-dibromo-4-(9-bromo-2,3-dimethyl-naphtho[2,3-b]thiophen-4-yl)-phenoxy]-4-phenyl-butyric acid;
(S)-2-[2,6-dibromo-4-(9-bromo-2,3-dimethyl-naphtho[2,3-b]thiophen-4-yl)-phenoxy]-4-phenyl-butyric acid;
2-[2,6-dibromo-4-(9-bromo-3-methyl-2-morpholin-4-ylmethyl-naphtho[2,3-b]thiophen-4-yl)-phenoxy]-3-phenyl-propionic acid; or
(R)-2-[2,6-dibromo-4-(2,3-dimethyl-9-phenylsulfanyl-naphtho[2,3-b]thiophen-4-yl)-phenoxy]-propionic acid; or a pharmaceutically acceptable salt or ester form thereof.

7. A method according to claim 1 wherein the protein-tyrosine phosphatase inhibitor compound is selected from the group of:
[2-bromo-4-(9-bromo-2,3-dimethyl-naphtho[2,3-b]thiophen-4-yl)-2-nitro-phenoxy]-3-phenyl-propionic acid;
2,6-dibromo-4-(9-bromo-2,3-dimethyl-naphtho[2,3-b]thiophen-4-yl)-phenol;
2-bromo-4-(9-bromo-2,3-dimethyl-naphtho[2,3-b]thiophen-4-yl)-6-nitro-phenol;
(R)-2-[2,6-dibromo-4-(9-bromo-2-diethylaminomethyl-3-methyl-naphtho[2,3-b]thiophen-4-yl)-phenoxy]-3-phenyl-propionic acid;
(R)-2-[2,6-dibromo-4-(2,3-dimethyl-naphtho[2,3-b]furan-4-yl)-phenoxy]-3-phenyl-propionic acid; or a pharmaceutically acceptable salt or ester form thereof.

8. A method according to claim 1 wherein the protein-tyrosine phosphatase inhibitor compound is selected from the group of:
(2R)-2-[4-9-bromo-2,3-dimethyl-naphtho[2,3-b]thiophen-4-yl)-2,6-diisopropyl-phenoxy]-3-phenyl-propionic acid;
(R)-2-[4-(9-bromo-2-,3-dimethyl-naphtho[2,3-b]thiophen-4-yl)-2,6-diethyl-phenoxy]-3-phenyl-propionic acid;
{(2R)-2-[4-(9-bromo-2,3-dimethyl-naphtho[2,3-b]thiophen-4-yl)-2,6-dimethyl-phenoxy]-3-phenyl-propionylamino}-acetic acid;
{(2R)-2-[4-(9-bromo-2,3-dimethyl-naphtho[2,3-b]thiophen-4-yl)-2,6-diethyl-phenoxy]-3-phenyl-propionylamino}-acetic acid;
(2R)-2-[4-(9-Bromo-2,3-dimethyl-naphtho[2,3-b]thiophen-4-yl)-phenoxy]-3-phenyl-propionic acid; or a pharmaceutically acceptable salt or ester form thereof.

9. A method of claim 1 wherein the protein-tyrosine phosphatase inhibitor compound is selected from the group of:
(2S)-2-[4-(9-Bromo-2-,3-dimethyl-naphtho[2,3-b]thiophen-4-yl)-2,6-dimethyl-phenoxy]-3-phenyl-propionic acid;
{(2R)-2-[4-(2,3-Dimethyl-naphtho[2,3-b]thiophen-4-yl)-2,6-diethyl-phenoxy]-3-phenyl-propionylamino}-acetic acid;
(R)-2-[4-(9-Bromo-2-,3-dimethyl-naphtho[2,3-b]furan-4-yl)-2,6-diethyl-phenoxy]-3-phenyl-propionic acid;
(R)-2-[2-Cyclopentyl-4-(2-,3-dimethyl-naphtho[2,3-b]thiophen-4-yl)-phenoxy]-propionic acid;
(R)-2-[4-(9-Bromo-2-,3-dimethyl-naphtho[2,3-b]thiophen-4-yl)-2-cyclopentyl-phenoxy]-propionic acid; or a pharmaceutically acceptable salt or ester form thereof.

10. A method of claim 1 wherein the protein-tyrosine phosphatase inhibitor compound is selected from the group of:
(R)-2-[4-(2-,3-Dimethyl-naphtho[2,3-b]thiophen-4-yl)-2-ethyl-phenoxy]-3-phenyl-propionic acid;
2-Bromo-4-(2-,3-dimethyl-naphtho[2,3-b]furan-4-yl)-6-ethyl-phenol;
(R)-2-[2-Bromo-4-(2-,3-dimethyl-naphtho[2,3-b]furan-4-yl)-6-ethyl-phenoxy]-3-phenyl-propionic acid;
(R)-2-[4-(9-Bromo-2-,3-dimethyl-naphtho[2,3-b]thiophen-4-yl)-2-propyl-phenoxy]-3-phenyl-propionic acid;
(2R)-2-[4-(9-Bromo-2-diethylaminomethyl-3-methyl-naphtho[2,3-b]thiophen-4-yl)-2,6-diisopropyl-phenoxy]-3-phenyl-propionic acid; or a pharmaceutically acceptable salt or ester form thereof.

11. A method of claim 1 comprising lowering a blood lipoprotein level in a mammal experiencing or subject to Syndrome X or type II diabetes, the method comprising administering to a mammal in need thereof a pharmaceutically effective amount of a protein-tyrosine phosphatase inhibitor and a pharmaceutically effective amount of an angiotensin converting enzyme inhibitor.

12. A method of claim 13 wherein the blood lipoprotein is low density lipoprotein.

13. A method of claim 1 comprising lowering a blood triglyceride level in a mammal experiencing or subject to Syndrome X or type II diabetes, the method comprising administering to a mammal in need thereof a pharmaceutically effective amount of a a protein-tyrosine phosphatase inhibitor and a pharmaceutically effective amount of an angiotensin converting enzyme inhibitor.

14. A method of claim 1 comprising lowering a free fatty acid level in a mammal experiencing or subject to Syndrome X or type II diabetes, the method comprising administering to a mammal in need thereof a pharmaceutically effective amount of a protein-tyrosine phosphatase inhibitor and a pharmaceutically effective amount of an angiotensin converting enzyme inhibitor.

15. A method of claim 1 comprising inhibiting atherosclerosis in a mammal experiencing or subject to type II diabetes.

16. A method of claim 1 wherein the antilipemic agent is a bile acid sequestrant agent

17. A method of claim 16 wherein the bile acid sequestrant agent is selected from colestipol or colesevelam, or a pharmaceutically acceptable salt form thereof.

18. A method of claim 1 wherein the antilipemic agent is a fibric acid derivative.

19. A method of claim 18 wherein the fibric acid derivative is selected from clifofibrate, gemfibrozil or fenofibrate, or a pharmaceutically acceptable salt form thereof.

20. A method of claim 1 wherein the antilipemic agent is an HMG-CoA reductase inhibitor.

21. A method of claim 20 wherein the HMG-CoA reductase inhibitor is selected from cerivastatin, fluvastatin, atorvastatin, lovastatin, pravastatin or simvastatin, or a pharmaceutically acceptable salt form thereof.

22. A method of claim 1 wherein the antilipemic agent is a nicotinic acid compound.

23. A method of claim 22 wherein the nicotinic acid compound is niacin.

24. A method of claim 1 wherein the antilipemic agent is a lipase inhibiting agent.

25. A method of claim 24 wherein the lipase inhibiting agent is orlistat.

26. A method for lowering the cardiovascular risk profile of mammal experiencing or subject to Syndrome X or type II diabetes, the method comprising administering to a mammal in need thereof a pharmaceutically effective amount of (2R)-2-[4-(9-Bromo-2,3-dimethyl-naptho[2,3-b]thiophen-4-yl)-2,6-dimethyl-phenoxy]-3-phenyl-propionic acid, or (R)-2-[2,6-Dibromo-4-(9-bromo-2,3-dimethyl-naphtho[2,3-b]thiophen-4-yl)-phenoxy]3-phenyl-propionic acid, or (R)-2-[4-(9-Bromo-2,3-dimethyl-naphtho[2,3-b]thiophen-4-yl)-2,6-diethyl-phenoxy]-3-phenyl-propionic acid, or a pharmaceutically acceptable salt or ester form thereof, and a pharmaceutically effective amount of an antilipemic agent.

27. A method of claim 26 wherein the lowering of the cardiovascular risk profile of a mammal experiencing or subject to type II diabetes comprises lowering a blood lipoprotein level in the mammal.

28. A method of claim 26 wherein the blood lipoprotein is low density lipoprotein.

29. A method of claim 26 wherein the lowering of the cardiovascular risk profile of a mammal experiencing or subject to type II diabetes comprises lowering a blood triglyceride level in the mammal.

30. A method of claim 26 wherein the lowering of the cardiovascular risk profile of a mammal experiencing or subject to type II diabetes comprises lowering a free fatty acid level in the mammal.

31. A method of claim 26 wherein the lowering of the cardiovascular risk profile of a mammal experiencing or subject to type II diabetes comprises inhibiting atherosclerosis in a mammal experiencing or subject to type II diabetes.

32. A method of claim 26 wherein the antilipemic agents is a bile acid sequestrant agent

33. A method of claim 32 wherein the bile acid sequestrant agent is selected from colestipol or colesevelam, or a pharmaceutically acceptable salt form thereof.

34. A method of claim 26 wherein the antilipemic agent is a fibric acid derivative.

35. A method of claim 34 wherein the fibric acid derivative is selected from clifofibrate, gemfibrozil or fenofibrate, or a pharmaceutically acceptable salt form thereof.

36. A method of claim 26 wherein the antilipemic agent is an HMG-CoA reductase inhibitor.

37. A method of claim 36 wherein the HMG-CoA reductase inhibitor is selected from cerivastatin, fluvastatin, atorvastatin, lovastatin, pravastatin or simvastatin, or a pharmaceutically acceptable salt form thereof.

38. A method of claim 26 wherein the antilipemic agent is a nicotinic acid compound.

39. A method of claim 38 wherein the nicotinic acid compound is niacin.
